(19) United States
(12) Patent Application Publication
(10) Pub. No.: US 2003/0176811 A1
(43) Pub. Date: Sep. 18, 2003

(54) Bone marrow extracting apparatus

(76) Inventor: Ira L. Shapira, Highland Park, IL (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/095,921

(22) Filed: Mar. 12, 2002

(51) Int. Cl. A61B 10/00

(52) U.S. Cl. 600/565

(57) ABSTRACT

Apparatus for extracting bone material from an extraction site using a vacuum source. The apparatus comprises a needle having a proximal end adapted for fluid communication with the vacuum source and a distal end. The needle has an interior surface defining a passageway extending from the proximal end to the distal end. An abrading member is disposed in the needle passageway, and an actuator is operably coupled to the abrading member.

### FIELD OF INVENTION

The present invention relates generally to methods and apparatus for recovering bone material, such as bone marrow, bone, and contiguous tissue, from a patient.

### BACKGROUND OF THE INVENTION

There are a number of diseases in which the bone marrow is defective, such as aplastic anemia, some forms of leukemia, and deficiencies in the bone marrow caused by cancer treatments with drugs and irradiation. The treatment of choice for these diseases is bone marrow transplantation, provided a genetically compatible donor can be found. For instance, bone marrow transplants are significantly reducing the death toll from childhood leukemias.

Bone marrow, also called myeloid tissue, is a soft, gelatinous tissue that fills the cavity of the bones. Human bone consists of a hard outer cortex and a soft medullary cavity that contains bone marrow. Bone marrow consists of stroma, or supporting tissues which have spaces packed by blood cells. Bone marrow is either red or yellow, depending upon the preponderance of vascular (red) or fatty (yellow) tissue. In humans, the red bone marrow forms all of the blood cells with the exception of the lymphocytes, which are produced in the marrow and reach their mature form in the lymphoid organs. Yellow bone marrow serves primarily as a storehouse for fats, but may be converted to red marrow under certain conditions, such as severe blood loss or fever. At birth, and until about the age of seven, all human marrow is red, as the need for new blood formation is high. Thereafter, fat tissue gradually replaces the red marrow, which in adults is found in the vertebrae, hips, breast bone, ribs, and skull, and at the ends of the long bones of the arms and legs, other cancellous, or spongy bones, and the central cavities of the long bones. In mammals, blood formation in adults takes place predominantly in the marrow. Because the white blood cells produced in the bone marrow are involved in the body's immune defenses, marrow transplants have been used to treat certain types of immune deficiencies. The sensitivity of marrow to damage by radiation and some anticancer drugs accounts for the tendency of these treatments to impair immunity.

Bone marrow transplants can be divided into three groups according to the source of the marrow for transplantation. They are called autologous, syngeneic, or allogeneic. Autologous transplantation means that the bone marrow has been received directly from the recipient, and will be an exact genetic match. A syngeneic transplant comes from an identical twin of the recipient and will also be an exact genetic match. However, for allogeneic transplants, the bone marrow is provided by another person, and the possibility of exact genetic matching is very low.

It is reported that approximately 12,000 bone marrow transplants were performed in 1992, approximately half of which were allogeneic and half autologous. Autologous transplantation has grown significantly during the past several years as improvements in procedures are made. The number of patients receiving allogeneic transplants is also rising due in large part because donor registries have increased the number of readily available donors. Advances in bone marrow transplantation techniques will likely continue to expand the use of the bone marrow transplant procedure.

Generally, the recipient's sibling or parent will serve as the best source as the donor because of the high possibility of genetic matching. However, there are many cases where neither the parent nor the sibling will be a compatible genetic match for the recipient. There has been a recent increase in the use of bone marrow from unrelated donors which can provide genetic compatibility between the donor and recipient. This increase has been made possible through the existence of large bone marrow registries, such as the National Marrow Donor Program, and the American Bone Marrow Donor Registry. The drawback to these registries are the insufficient number of donors that genetically match closely enough with potential recipients to be of use.

The success of the bone marrow transplantation technique depends heavily on genetically cross-matching the donor marrow cells to those of the recipient to prevent rejection. There is a significant tendency for the recipient patient to reject an allografted marrow because parts of the donor marrow will attack their new host. There is an additional hazard because immune system cells in a marrow graft can react against the patient's tissues, causing serious and sometimes fatal graft versus host disease. The ability to accept a bone marrow transplant (graft) from a donor, is dependent on the recipient sharing all of the donor's histocompatibility genes. To avoid graft versus host rejection in the past, special immunosuppressive treatment has been given. The use of monoclonal antibodies to selectively remove harmful lymphocytes from the donor marrow has been successful in some cases to prevent graft versus host disease. However, the risk remains that unless the bone marrow source is from the patient himself, an identical twin, sibling, parent, or other genetically compatible donor, that the bone marrow transplantation cannot take place because it will result in graft versus host rejection, and the failure of the treatment, and possibly the death of the recipient.

Therefore, there is a significant need to collect and store genetically compatible bone marrow for use in cases where bone marrow transplantation is necessary to save the life of an individual. Because of the significant possibility that a donor cannot be found which is a close genetic match to the recipient, there is a need to collect and store an individual's own bone marrow while that individual is still healthy. If this is done, there will be a complete genetic match, and the dangers of graft versus host rejection will be eliminated which increases the success of the treatment.

The collection of bone marrow for transplantation purposes is usually accomplished by inserting a needle into a donor's hip or pelvic bone. Several small incisions are made in the pelvic area, and the needle is inserted through these incisions approximately 25 to 30 times to withdraw the bone marrow from the bones. The extraction process typically lasts at least one hour or more, or until approximately 500 to 1000 milliliters of the donor's marrow is withdrawn.

The donor will fully recover in approximately a few weeks when all the donated marrow has been replaced within the body. However, the extraction process is painful and there is typically soreness around the incisions until healing can occur. Typically, the donors also feel fatigued for some time after the procedure. The side effects to having donated bone marrow can vary from donor to donor. Infection from the incision is always a possibility. Additionally, blood loss can also occur, and proper medical attention is required. It is recommended that donors routinely store supplies of their own blood for infusion during and after the extraction procedure in cases of emergencies.

Bone marrow can be obtained through biopsy or aspiration from the sternum or the calvarium in adults, and in long bones, such as the femur and tibia, in adolescents. Biopsy needles for extraction of solid bone marrow are known. Examples of such biopsy needles are U.S. Pat. Nos. 2,426,535; 2,496,111; 4,266,555; 4,543,966; 4,487,209; 4,840,184; and 4,922,602, which show the overall structure and orientation of the components. Needles used for aspiration of liquid bone marrow are disclosed in U.S. Pat. No. 4,469,109. Needles designed to both biopsy and aspirate bone marrow are disclosed in U.S. Pat. Nos. 2,496,111; 3,587,560; 5,012,818; and 5,357,974.

In the known bone marrow extraction devices, bone marrow is drawn into the needle under partial vacuum pressure. To create the partial vacuum pressure inside the needle, the devices are typically adapted to engage a syringe at a distal end of the needle. While such devices are suitable for extracting bone marrow having lower viscosities, they are inefficient at, if not incapable of, extracting bone marrow having higher viscosities.

### BRIEF DESCRIPTION OF THE DRAWINGS

FIG. 1 is a schematic side elevational view of one embodiment of extraction apparatus in accordance with the teachings of the present invention;

FIG. 2 is an enlarged sectional view of a bone marrow of the extraction apparatus of FIG. 1; and

FIG. 3 is an enlarged bottom end view taken along line 3-3 of FIG. 2.

### DETAILED DESCRIPTION

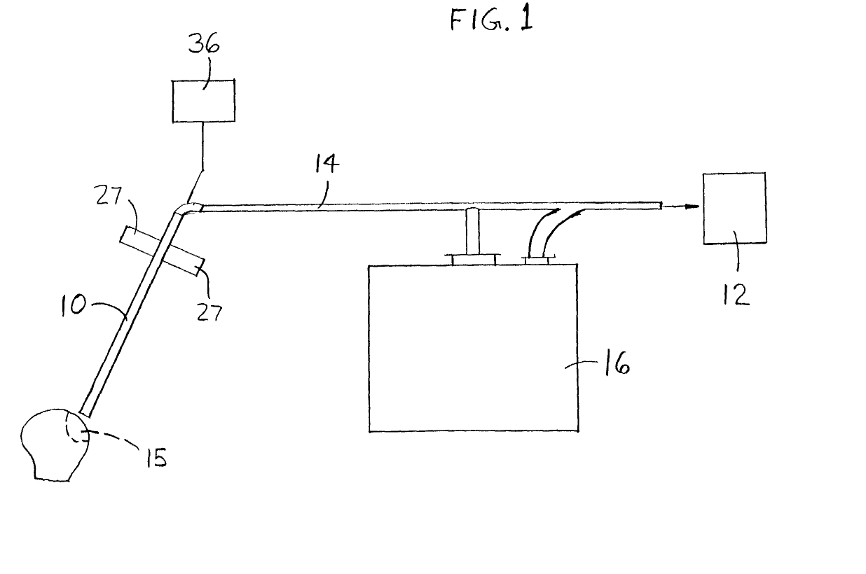

One embodiment of bone material extraction apparatus in accordance with the teachings of the present invention is illustrated at FIG. 1. The apparatus may include a bone marrow needle 10 connected to a vacuum source 12 by a suction tube 14. The vacuum source may include a pump, vacuum blower, syringe, or other device capable of generating a partial vacuum pressure. A collection chamber 16 may also be connected to the suction tube 14. In operation, the bone marrow needle 10 is positioned at an extraction site 15 to remove bone material from a patient. The bone material may be pulled through the suction tube 14 and be deposited in the collection chamber 16.

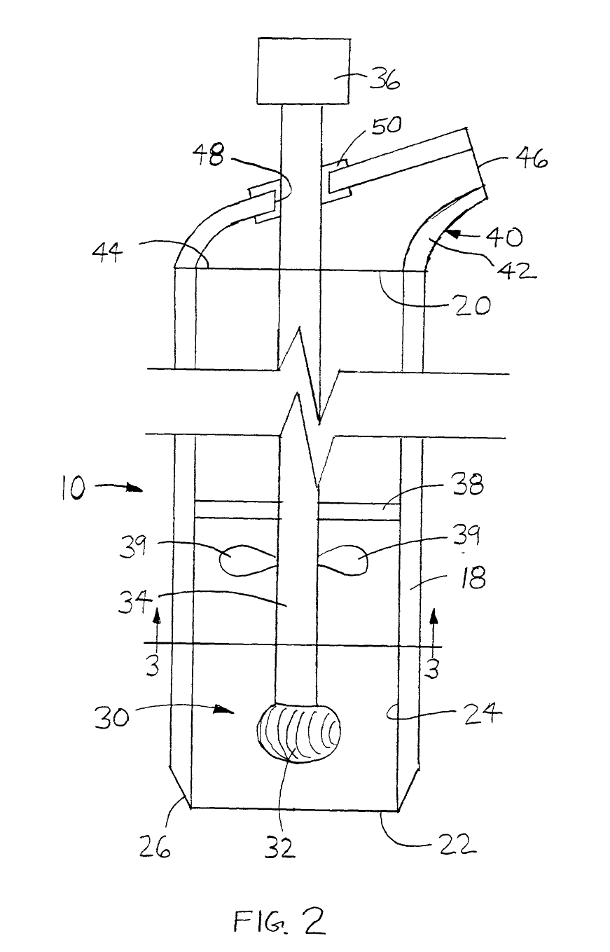

The bone marrow needle 10 is illustrated in greater detail at FIG. 2. The needle 10 includes a hollow shaft 18 having a proximal end 20 adapted for fluid communication with the suction tube 14 and a distal end 22. An interior wall of the shaft 18 defines a passageway 24 extending from the distal end 22 to the proximal end 20. The distal end 22 is formed as a knife edge 26 to assist in cutting through bone material. Handles 27 (FIG. 1) may be attached to the shaft 18 for gripping the needle 10 during use. The needle 10 may be adapted to receive a stylet (not shown) for use during insertion of the needle into the patient, as is generally known in the art.

While one type of bone marrow needle has been illustrated herein, it will be appreciated that the present invention may be used in any type of biopsy or aspiration needle known in the art. For example, the needle may have two passageways (or “lumens”), or may be used with a modified stylet for improved cutting. The prior art patents noted above describe some of the needles that may be used without departing from the teachings of the present invention.

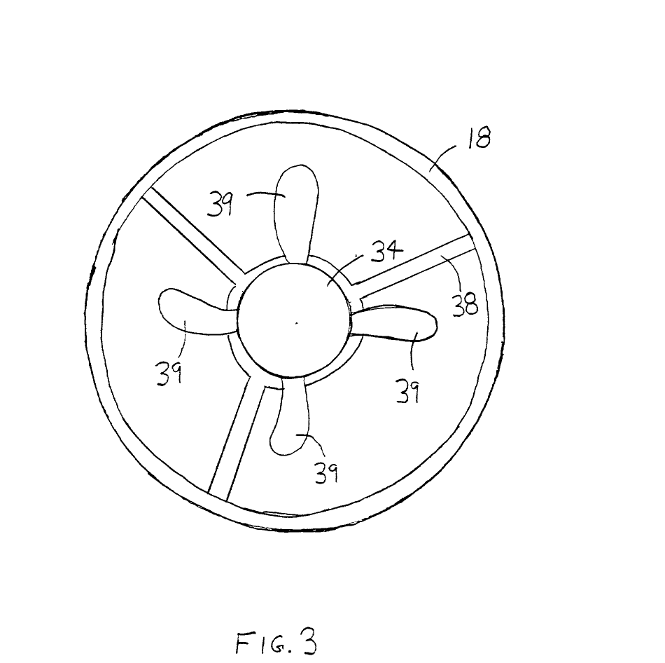

As best shown in FIG. 2, an abrading member 30 may be inserted inside the hollow shaft 18 for breaking up and/or liquifying bone material. To accommodate the abrading member 30, the stylet is removed from the needle 10 after the proximal end 20 is positioned at a bone material extraction site. According to the illustrated embodiment, the abrading member 30 includes a bur head 32 connected to a shank 34. The shank 34 is operatively coupled to an actuator, such as motor 36 (FIG. 1), for driving the bur head 32 in an abrading motion, such as by rotation, reciprocation, or otherwise. The bur head 32 may be positioned near the distal end 22 of the needle 10 so that it is located near the extraction site. A support ring 38 may extend from the shank 34 to the interior surface of the hollow shaft 18 for stabilizing the bur head 32. Cutter blades 39 may be attached to the shank 34 for breaking up bone material into smaller particles, as illustrated in FIGS. 2 and 3.

While the bur head 32 is illustrated herein as attached to a rigid shank 34, the shank may instead be flexible. The bur head 32 may also be permanently or removably attached to the shank 34. Instead of a bur head, the abrading member 30 may be provided as a file, such as a K-file (twisted wire), a Hedstrom file (cut wire), or a reciprocating file, as a cutting blade, or any other known abrading structure. The motor 36 attached to the abrading member 30 may be electrical or pneumatic, and may generate a rotational, vibrational, reciprocating, or other movement for driving the abrading member. The actuator may also be provided as an ultrasonic wave generator or any other known means for actuating the abrading member.

An adapter 40 may be attached to the proximal end 20 for connecting the needle 10 to the suction tube 14. As shown in FIG. 2, the adapter 40 includes a side wall 42 having a first end 44 in sealing engagement with the needle proximal end 20 and a second end 46 adapted for connection to the suction tube 14. A port 48 is formed in the sidewall and sized to allow the shank 34 to pass therethrough. A gasket 50 may be inserted in the port 48 and may seal with the shank 34 to prevent leakage.

In operation, the needle 10 extracts bone material from the extraction site. The extraction site may be formed in any part of the body using the needle 10 with the stylet (not shown) inserted therein, as is generally known in the art. The stylet is subsequently removed and the abrading member 30 inserted into the needle 10. The vacuum source 12 creates a partial vacuum that is transferred through the suction tube 14 to the needle passageway 24. The partial vacuum is generally sufficient to draw liquid or low viscosity bone material into the needle 10, but is incapable or does not efficiently extract solid or high viscous bone material. The abrading member 30, however, serves to break up and liquify bone material.

The extraction site may be formed in any area from which bone material may be extracted. Accordingly, the extraction site may be formed in the jaw, sternum, skull, hip, or any other area. Furthermore, as used herein, the phrase ‘bone material’ is intended to include various hard bone materials (such as cortical bone), soft bone materials (such as marrow or liquid materials), and contiguous tissue (such as dental pulp).

In an exemplar embodiment, the extraction apparatus effects the removal of bone material from a donor and mixes the removed bone marrow with a suitable form of solution, such as a mixture of anticoagulant and saline or electrolytic solution. The bone marrow and bone marrow fluid removed from the donor are then transferred either into a cell separator or a suitable collection bag, such as the collection chamber 16, so as to permit separation of the bone material and fluid for subsequent processing and long-term storage. The collected bone material may also be used for the subsequent reinjection into the donor or into other donee patients in future medical procedures, such as bone marrow transplantation.

In the removal of the bone material from the donor, a solution consisting of heparin or other anticoagulant compositions, together with a saline solution, can be mixed with the bone marrow and bone marrow fluid before, during, and/or after being transferred into separating or collecting means. In addition, a preservation solution may be mixed with the bone material at any time to extend the storage life of the bone material.

The collection chamber 16 may be a bag containing chemicals for preserving bone material. The chemicals may be in the bag prior to the withdrawal of bone material from the patient. In this manner, after bone material has been collected, the chamber 16 can be stored cold directly. Additionally or alternatively, chemicals can be added to the chamber 16 during or after collection of bone material to preserve the bone material. Suitable means for adding chemicals to a container such as the chamber 16 are well known in the art and may include penetrable membranes at specific locations on the chamber 16. The chamber 16 is preferably collapsible so that air may be removed after collection has occurred. Removal of air increases the useful life of the bone marrow.

The foregoing detailed description has been given for clearness of understanding only, and no unnecessary limitations should be understood therefrom, as modifications will be obvious to those skilled in the art.

What is claimed is:

1. Apparatus for extracting bone material from an extraction site using a vacuum source, the apparatus comprising:
a needle having a proximal in fluid communication with the vacuum source and a distal end, the needle having an interior surface defining a passageway extending from the proximal end to the distal end;
an abrading member disposed in the needle passageway; and
an actuator operably coupled to the abrading member.

2. The apparatus of claim 1, in which the abrading member comprises a bur.

3. The apparatus of claim 1, in which the actuator comprises a motor.

4. The apparatus of claim 1, in which the abrading member comprises a shank coupled to the actuator and an abrading head attached to the shank.

5. The apparatus of claim 4, in which a cutter blade is attached to the shank.

6. The apparatus of claim 1, further comprising an adapter having a first end sealingly engaging the needle proximal end and a second end in fluid communication with the vacuum source.

7. The apparatus of claim 6, in which a suction tube is connected between the adapter second end and the vacuum source.

8. The apparatus of claim 7, in which a collection chamber is attached to the suction tube.

9. A method of extracting bone material from a bone, the method comprising:
forming a bone material extraction site in the bone;
inserting a bone material needle into the extraction site;
placing the bone material needle under partial vacuum pressure to draw bone material into the needle; and
abrading the bone material drawn into the needle to reduce a viscosity of the bone material, thereby increasing the amount of bone material extracted from the extraction site.

10. The method of claim 9, in which an abrading member is used to abrade the bone material drawn into the needle.

11. The method of claim 10, in which the abrading member comprises a bur.

12. The method of claim 10, in which the abrading member comprises a shank attached to an abrading head.

13. The method of claim 12, in which a cutter blade is attached to the shank.

14. The method of claim 9, further comprising collecting abraded bone material in a collection chamber.

15. The method of claim 9, in which the needle is used to form the extraction site.
